(19) United States
(12) Patent Application Publication
(10) Pub. No.: US 2012/0053421 A1
(43) Pub. Date: Mar. 1, 2012

(54) LIGHT GUIDE FOR AN ENDOSCOPE, ENDOSCOPE EQUIPPED WITH THE LIGHT GUIDE, AND METHOD FOR PRODUCING THE LIGHT GUIDE FOR AN ENDOSCOPE

(75) Inventors: Koji YOSHIDA, Ashigarakami-gun (JP); Tatsuya Yoshihiro, Ashigarakami-gun (JP)

(73) Assignee: FUJIFILM CORPORATION, Tokyo (JP)

(21) Appl. No.: 13/221,751

(22) Filed: Aug. 30, 2011

(30) Foreign Application Priority Data
Aug. 31, 2010 (JP) 193798/2010

(51) Int. Cl. A61B 1/06; B23P 11/00

(52) U.S. Cl. 600/182; 29/428

(57) ABSTRACT

A light guide for an endoscope is equipped with an optical fiber. The optical fiber has an input side tapered portion and an output side tapered portion. The input side tapered portion is a predetermined portion of the optical fiber that includes an input end into which the illumination light enters the optical fiber, and the core of the optical fiber having a shape that becomes thicker toward the input end in the light input side tapered portion. The light output side tapered portion is a predetermined portion of the optical fiber that includes an output end through which the illumination light is output, and the core of the optical fiber having a shape that becomes thicker toward the output end in the light output side tapered portion. The core diameter at the input end is greater than the core diameter at the output end.

### BACKGROUND OF THE INVENTION

1. Field of the Invention

The present invention is related to a light guide for an endoscope which is inserted into body cavities and guides illumination light to a portion to be observed, and an endoscope equipped with the light guide. The present invention is also related to a method for producing a light guide for an endoscope.

2. Description of the Related Art

Endoscope systems for observing tissue within body cavities are widely known. For example, endoscope systems that obtain visible images by imaging using white light to illuminate portions to be observed within body cavities and display the visible images on screens of monitors are in wide practical use.

Light guides for endoscopes equipped with optical fibers for guiding illumination light into body cavities are utilized in the aforementioned endoscope systems. A laser light source may be utilized as the light source for generating the illumination light. Laser beams output from laser light sources have high directionality. For this reason, traditionally, predetermined portions in the vicinities of output ends of optical fibers are formed as tapered shapes that become thinner toward the ends in order to increase the angle of spread for use as the illumination light, as disclosed in Japanese Unexamined Patent Publication No. 2009-297188.

However, forming the output end of an optical fiber to be thin as disclosed in Japanese Unexamined Patent Publication No. 2009-297188 causes problems from the viewpoint of safety standards for laser beams. There are cases that laser beams emitted by laser light sources will be harmful to human bodies due to their high power densities per visual angle, even if the emission amount is low. Accordingly, in the case that a laser light source is utilized as an illumination light source, it is preferable to use a laser having the lowest level safety standard class as possible, from the viewpoint of safety of operating sites.

### SUMMARY OF THE INVENTION

The present invention has been developed in view of the foregoing circumstances. It is an object of the present invention to provide a light guide for an endoscope that realizes an increase in angle of spread without tapering an optical fiber such that the optical fiber becomes thinner at the end thereof, and also enables reduction of laser beam intensity per visual angle at an output end of the optical fiber by forming a light source having a large light emitting area. It is another object of the present invention to provide an endoscope equipped with the above light guide.

It is a further object of the present invention to provide a method for producing a light guide for an endoscope that enables production of a light guide for an endoscope that exhibits the aforementioned operational effects and a method for producing an endoscope equipped with such a light guide.

Alight guide for an endoscope of the present invention that achieves the above object is equipped with an optical fiber for guiding illumination light to a portion to be observed, the optical fiber comprising:

a light input side tapered portion; and

a light output side tapered portion;

the light input side tapered portion being a predetermined portion of the optical fiber that includes an input end into which the illumination light enters the optical fiber, and the core of the optical fiber having a shape that becomes thicker toward the input end in the light input side tapered portion;

the light output side tapered portion being a predetermined portion of the optical fiber that includes an output end through which the illumination light is output, and the core of the optical fiber having a shape that becomes thicker toward the output end in the light output side tapered portion; and

the core diameter at the input end being greater than the core diameter at the output end.

In the light guide for an endoscope of the present invention, it is preferable for the ratio of the core diameter at the input end with respect to the core diameter at the output end to be within a range from 2 to 3.

It is also preferable for the core diameter at the input end to be within a range from 300 μm to 6000 μm, and for the core diameter at the output end to be within a range from 150 μm to 4000 μm. The lower limits of the core diameters are limited by the size of the light source which is considered to be a point light source from the viewpoint of laser safety. The upper limits of the core diameters are limited by core diameters that enable the degree of flexibility generally required of light guides for endoscopes to be obtained.

Further, it is preferable for the optical fiber to be a multimode optical fiber.

An endoscope of the present invention comprises:

a light guide for an endoscope as described above;

a light source for generating the illumination light, connected to an input side of the light guide for an endoscope; and

an imaging section, for receiving light generated at the portion to be observed due to irradiation of the illumination light guided by the light guide for an endoscope and for imaging the portion to be observed.

In the present specification, the phrase “light generated at the portion to be observed due to irradiation of the illumination light” refers to reflected light in the case that a visible image is obtained using white light as the illumination light, and refers to fluorescence corresponding to excitation light in the case that a fluorescence image is obtained using the excitation light as the illumination light, for example.

A first method for producing a light guide for an endoscope that guides illumination light to a portion to be observed of the present invention comprises:

preparing a first optical fiber having a first tapered portion at a first end thereof and a second optical fiber having a second tapered portion at a first end thereof;

fusing a second end of the first optical fiber and a second end of the second optical fiber to form a third optical fiber having the first tapered portion and the second tapered portion; and

placing the third optical fiber into a probe portion of an endoscope such that the second tapered portion is the input side and the first tapered portion is the output side of the illumination light;

the first tapered portion being a predetermined portion of the first optical fiber that includes the first end thereof, and the core of the optical fiber having a shape that becomes thicker toward the first end of the first optical fiber in the predetermined portion;

the second tapered portion being a predetermined portion of the second optical fiber that includes the first end thereof, and

the core of the optical fiber having a shape that becomes thicker toward the first end of the second optical fiber in the predetermined portion; and

the core diameter at the first end of the second optical fiber being greater than the core diameter of the first end of the first optical fiber.

In the first method for producing a light guide for an endoscope of the present invention, it is preferable for the ratio of the core diameter at the first end of the first optical fiber with respect to the core diameter at the first end of the second optical fiber to be within a range from 2 to 3.

Further, it is preferable for the core diameter at the first end of the second optical fiber to be within a range from 300 μm to 6000 μm, and for the core diameter at the first end of the first optical fiber to be within a range from 150 μm to 4000 μm.

A second method for producing a light guide for an endoscope that guides illumination light to a portion to be observed of the present invention comprises:

processing a predetermined portion of an optical fiber that includes a first end of the optical fiber to be tapered such that the core of the optical fiber in the predetermined portion becomes thicker toward the first end;

processing a predetermined portion of the optical fiber that includes a second end of the optical fiber to be tapered such that the core of the optical fiber in the predetermined portion becomes thicker toward the second end and such that the core diameter at the second end is greater than the core diameter at the first end; and

placing the optical fiber within a probe portion of an endoscope such that the end having the greater core diameter is the light input side and the end having the smaller core diameter is the output side of the illumination light.

In the present specification, to perform a tapering process “such that the core diameter at the second end is greater than the core diameter at the first end” refers to various cases. Such cases include: a case in which a tapering process is administered on the first end first, and then a tapering process is administered on the second end such that the core diameter at the second end is greater than that of the first end; and a case in which a tapering process is administered on the second end first, and then a tapering process is administered on the first end such that the core diameter at the first end is less than that of the second end, causing the core diameter at the second end to be greater as a result. That is, the second method for producing a light guide for an endoscope of the present invention does not depend on the order in which the tapering processes are administered onto the ends of the optical fiber.

In the second method for producing a light guide for an endoscope of the present invention, it is preferable for the ratio of the core diameter at the second end of the optical fiber with respect to the core diameter at the first end of the optical fiber to be within a range from 2 to 3.

Further, it is preferable for the core diameter at the second end of the optical fiber to be within a range from 300 μm to 6000 μm, and for the core diameter at the first end of the optical fiber to be within a range from 150 μm to 4000 μm.

The light guide for an endoscope of the present invention and the endoscope equipped with the light guide are equipped with an optical fiber. The optical fiber has a light input side tapered portion and a light output side tapered portion. The light input side tapered portion is a predetermined portion of the optical fiber that includes an input end into which the illumination light enters the optical fiber, and the core of the optical fiber has a shape that becomes thicker toward the input end in the light input side tapered portion. The light output side tapered portion is a predetermined portion of the optical fiber that includes an output end through which the illumination light is output, and the core of the optical fiber has a shape that becomes thicker toward the output end in the light output side tapered portion. The optical fiber is characterized by the core diameter at the input end being greater than the core diameter at the output end. Thereby, laser beam intensity per visual angle can be reduced by increasing the light emitting area at the output end of the optical fiber. Meanwhile, the input end of the optical fiber is formed as a tapered shape that becomes thicker toward the input end, which has a greater core diameter than that of the output end. Therefore, the angle of spread at the output end can be increased, according to the conservation of etendue. As a result, an increase in angle of spread can be realized without tapering the optical fiber such that the optical fiber becomes thinner at the end thereof. At the same time, reduction of laser beam intensity per visual angle at an output end of the optical fiber can be realized by forming a light source having a large light emitting area.

Further, based on the effects described above, the light guide for an endoscope of the present invention and the endoscope equipped with the light guide exhibit the advantageous effects that an illuminated region is expanded and the safety standard class of laser light is reduced.

The first method for producing a light guide for an endoscope of the present invention is a method for producing a light guide for an endoscope that guides illumination light to a portion to be observed, and comprises: preparing a first optical fiber having a first tapered portion at a first end thereof and a second optical fiber having a second tapered portion at a first end thereof; fusing a second end of the first optical fiber and a second end of the second optical fiber to form a third optical fiber having the first tapered portion and the second tapered portion; and placing the third optical fiber into a probe portion of an endoscope such that the second tapered portion is the input side and the first tapered portion is the output side of the illumination light; the first tapered portion being a predetermined portion of the first optical fiber that includes the first end thereof, and the core of the optical fiber having a shape that becomes thicker toward the first end of the first optical fiber in the predetermined portion; the second tapered portion being a predetermined portion of the second optical fiber that includes the first end thereof, and the core of the optical fiber having a shape that becomes thicker toward the first end of the second optical fiber in the predetermined portion; and the core diameter at the first end of the second optical fiber being greater than the core diameter of the first end of the first optical fiber. Thereby, it becomes possible to produce a light guide for an endoscope that exhibits the aforementioned operational effects and an endoscope equipped with the light guide.

The second method for producing a light guide for an endoscope that guides illumination light to a portion to be observed of the present invention comprises: processing a predetermined portion of an optical fiber that includes a first end of the optical fiber to be tapered such that the core of the optical fiber in the predetermined portion becomes thicker toward the first end; processing a predetermined portion of the optical fiber that includes a second end of the optical fiber to be tapered such that the core of the optical fiber in the predetermined portion becomes thicker toward the second end and such that the core diameter at the second end is greater than the core diameter at the first end; and placing the optical fiber within a probe portion of an endoscope such that the end having the greater core diameter is the light input side and the end having the smaller core diameter is the output side of the illumination light. Thereby, it becomes possible to produce a light guide for an endoscope that exhibits the aforementioned operational effects and an endoscope equipped with the light guide.

### BRIEF DESCRIPTION OF THE DRAWINGS

FIG. 1 is a diagram that illustrates the outer appearance of an endoscope system that employs a light guide for an endoscope of the present invention.

FIG. 2 is a diagram that schematically illustrates the inner structures of the endoscope system that employs the light guide for an endoscope of the present invention.

FIG. 3 is a sectional diagram that schematically illustrates an optical fiber having both ends thereof tapered, for use in the light guide for an endoscope of the present invention.

FIG. 4 is a collection of conceptual diagrams that illustrate the relationship between incident angles φ and angles of spread θ of output light, in optical fibers having predetermined tapered shapes.

FIG. 5 is a collection of sectional diagrams that illustrate optical fibers having tapered portions at both ends thereof, produced as embodiments of the present invention.

### DESCRIPTION OF THE PREFERRED EMBODIMENTS

Hereinafter, embodiments of the present invention will be described with reference to the attached drawings. However, the present invention is not limited, to the embodiments to be described below. Note that the dimensions, scale, etc. of constituent elements in the drawings may differ from the actual dimensions, scale, etc., in order to facilitate visual understanding.

[Embodiments of Light Guide for an Endoscope, Endoscope Equipped with the Light Guide, and Method for Producing the Light Guide for an Endoscope]

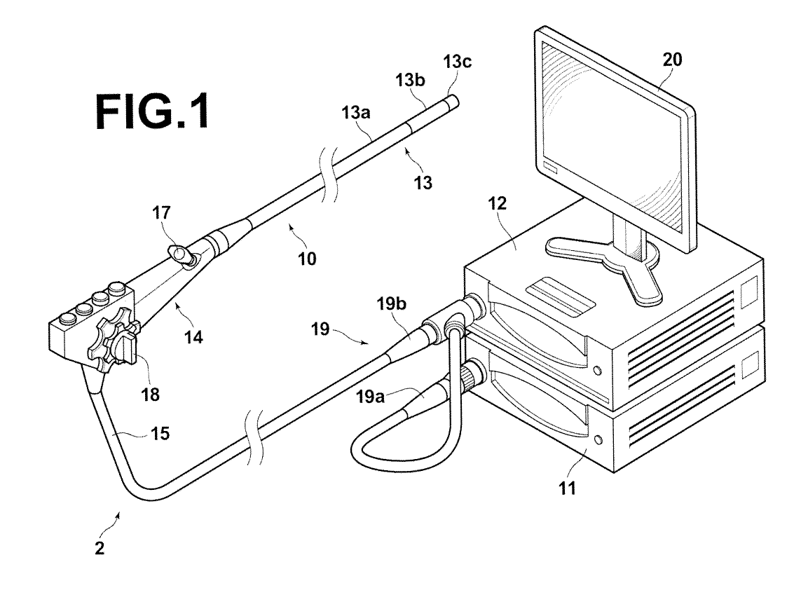

A light guide for an endoscope and an endoscope equipped with the light guide are utilized in an endoscope system 2 such as that illustrated in FIG. 1. As illustrated in FIG. 1, the endoscope system 2 is constituted by: an electronic endoscope 10 for obtaining images of portions to be observed within the bodies (body cavities) of subjects; a processing apparatus 11 for generating endoscope images; and a light source apparatus 12 that supplies illumination light for illuminating the interiors of the body cavities. A monitor 20 for displaying the endoscope images is connected to the processing apparatus 11.

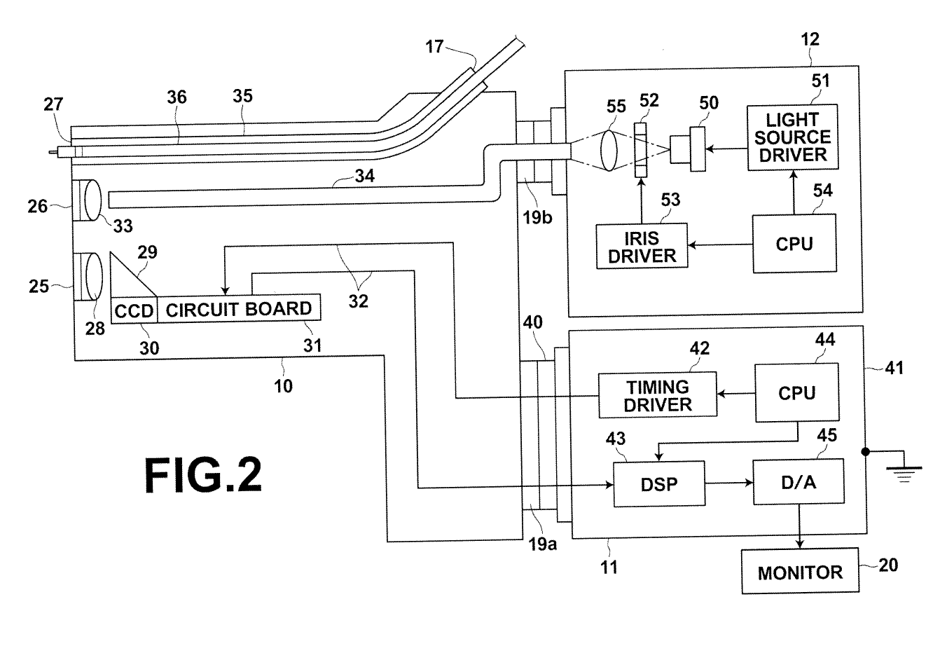

The electronic endoscope 10 is equipped with: a probe portion 13 to be inserted into body cavities; an operating portion 14 which is provided at the base end of the probe 13; and a universal cord 15 that extends from the operating portion 14. The probe portion 13 is constituted by: a long thin flexible tube portion 13a; a bight portion formed by a plurality of linked bight pieces; and a tip portion 13c positioned at the distal end of the probe portion 13. The tip portion 13c is formed by a rigid metal material or the like, and houses a CCD 30 (refer to FIG. 2) or the like for obtaining images within the body cavities therein.

The operating portion 14 is equipped with a forceps opening 17, an angle knob 18, and the like. The forceps opening 17 is linked to a forceps outlet 27 (refer to FIG. 2) formed in the tip portion 13c. Treatment implements are inserted through the forceps opening 17, and caused to protrude into the body cavities through the forceps outlet 27. The angle knob 18 is connected to the bight portion 13b via wires provided within the probe portion 13. The bight portion 13b bends to moves in the vertical and horizontal directions by pushing and pulling the wires by manipulating the angle knob 18. Thereby, the tip portion 13c can be oriented toward desired directions within the body cavities.

A connector 19 is provided at the extended end of the universal cord 15. The connector 19 is a combination type connector constituted by a communications connector 19a and a light source connector 19b, and is capable of being removably connected to the processing apparatus 11 and the light source apparatus 12.

As illustrated in FIG. 2, an observation window 25 for receiving image light of subjects, an illuminating window 26 through which the illumination light is irradiated, and the forceps outlet 27 are provided on the distal end surface of the tip portion 13c of the electronic endoscope 10. A light guiding optical system 28 and a prism 29 are provided to the rear of the observation window 25. The CCD 30 is provided directly beneath the prism 29, and the COD 30 is connected to a circuit board 31. Image light of subjects which has passed through the light guiding optical system 28 and the prism 29 enters a light receiving surface of the CCD 30. The CCD 30 outputs image signals based on the image light that enters the light receiving surface, and inputs the image signals to the circuit board 31.

The circuit board 31 is connected to a timing/driver circuit 42 and a digital signal processing circuit 43 (DSP 43) of the processing apparatus 11 via signal cables 32. The circuit board 31 is equipped with an analog signal processing circuit (not shown). The analog signal processing circuit administers a correlated double sampling process on the image signals input from the CCD 30, to remove reset noise and amplifier noise. Then, the image signals, from which noise has been removed, are amplified at a predetermined gain then converted to digital signals having a predetermined number of bits. The digital image signals are input to the DSP 43 of the processing apparatus 11 via the signal cables 32.

An irradiating lens 33 that irradiates the illumination light toward the interior of the body cavity is provided behind the illuminating window 26. The irradiating lens 33 faces the output end of a light guide 34. The light guide 34 penetrates through the probe portion 13, the operating portion 14, and the interior of the universal cord 15, and an input facet thereof protrudes out of the end of the light source connector 19b. When the light source connector 19b is connected to the light source apparatus 12, the input facet of the light guide 34 is inserted into the interior of the light source apparatus 12. The illumination light from the light source apparatus 12 is guided to the tip portion 13c by the light guide 34, and irradiated into the interior of the body cavities via the irradiating lens 33 and the illuminating window 26.

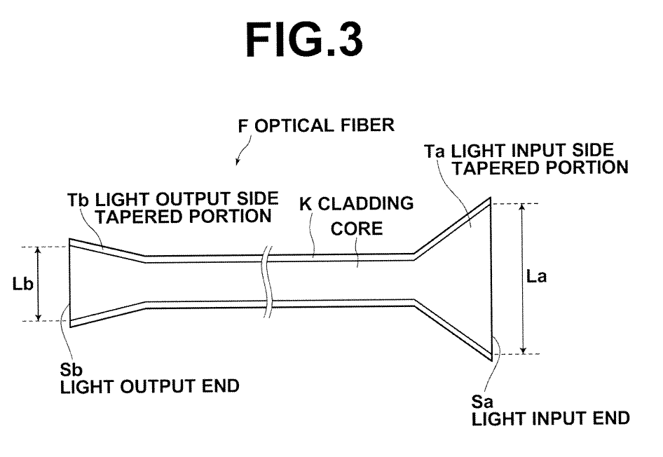

An optical fiber F that functions as a light guiding member of the light guide 34 is constituted by a core C and a cladding K. As illustrated in FIG. 3, the optical fiber F has a light input side tapered portion Ta and a light output side tapered portion Tb. The light input side tapered portion Ta is a predetermined portion of the optical fiber F that includes an input end Sa into which the illumination light enters the optical fiber F, and the core C of the optical fiber F has a shape that becomes thicker toward the input end Sa in the light input side tapered portion Ta. The light output side tapered portion Tb is a predetermined portion of the optical fiber F that includes an output end Sb through which the illumination light is output, and the core C of the optical fiber F having a shape that becomes thicker toward the output end Sb in the light output side tapered portion Tb.

The optical fiber F is formed such that a core diameter La at the light input end Sa is greater than a core diameter Lb at the light output end Sb. It is preferable for the ratio of the core diameter at the input end with respect to the core diameter at the output end to be within a range from 2 to 3. In addition, it is preferable for the core diameter at the input end to be within a range from 300 μm to 6000 μm, and the core diameter at the output end to be within a range from 150 μm to 4000 μm. Here, in the visible light and the near infrared light regions, optical fibers that satisfy the aforementioned conditions regarding the core diameters operate as multimode optical fibers.

The optical fiber F having tapered portions at both ends thereof can be formed by the following method, for example. First, a first optical fiber having a first tapered portion at a first end thereof and a second optical fiber having a second tapered portion at a first end thereof are prepared. Here, the first tapered portion is a predetermined portion of the first optical fiber that includes the first end thereof, and the core of the optical fiber has a shape that becomes thicker toward the first end of the first optical fiber in the predetermined portion. The second tapered portion is a predetermined portion of the second optical fiber that includes the first end thereof, and the core of the optical fiber has a shape that becomes thicker toward the first end of the second optical fiber in the predetermined portion. In addition, the core diameter at the first end of the second optical fiber is greater than the core diameter of the first end of the first optical fiber. Next, a second end of the first optical fiber and a second end of the second optical fiber are fused to form a third optical fiber having the first tapered portion and the second tapered portion. Thereby, the third optical fiber, which is an optical fiber as described above, is formed. Further, a plurality of such optical fibers are prepared by repeating the above steps as necessary. Thereafter, the third optical fiber is placed in a probe portion of an endoscope such that the second tapered portion is the input side and the first tapered portion is the output side of the illumination light, to obtain the light guide of the first embodiment.

Alternatively, the optical fiber F having the tapered portions at both ends thereof may be formed by the following method. First, a predetermined portion of an optical fiber that includes a first end of the optical fiber is processed to be tapered such that the core of the optical fiber in the predetermined portion becomes thicker toward the first end. Next, a predetermined portion of the optical fiber that includes a second end of the optical fiber is processed to be tapered such that the core of the optical fiber in the predetermined portion becomes thicker toward the second end and such that the core diameter at the second end is greater than the core diameter at the first end. At this time, the tapered portion having the larger core diameter may be formed first or second. An optical fiber as described above is formed by the steps above. Further, a plurality of such optical fibers may be prepared by repeating the above steps as necessary. Then, the optical fiber is placed within a probe portion of an endoscope such that the end having the greater core diameter is the light input side and the end having the smaller core diameter is the output side of the illumination light, to obtain a light guide of the first embodiment. In the method above, the tapering process may be executed by adjusting the speed at which the optical fiber is drawn. Commonly, optical fiber drawing apparatuses are capable of adjusting drawing speeds within a range from approximately 1000 m/min to 2600 m/min. Therefore, drawing can be performed such that the core diameters of the tapered portions and the core diameter of a linear portion can assume desired values.

In the case that the two ends of the optical fiber F are formed as tapered shapes that become thicker toward the ends and the core diameter of the input side is greater than the core diameter of the output side, increase in the angle of spread can be realized without tapering the end of an optical fiber to become thinner toward the end. In addition, a light source having a large light emitting area can be formed, thereby decreasing the laser beam intensity per visual angle at the output end of the optical fiber.

Further, the light guide of the present invention needs only to have the tapered shapes that become thicker toward the ends only at the two ends thereof. That is, it is possible to employ an optical fiber which has a thin diameter except at the two ends thereof. This is advantageous in that a thin diameter optical fiber superior in flexibility and rigidity can be utilized at the central portion of the light guide, at which bending deformations occur frequently.

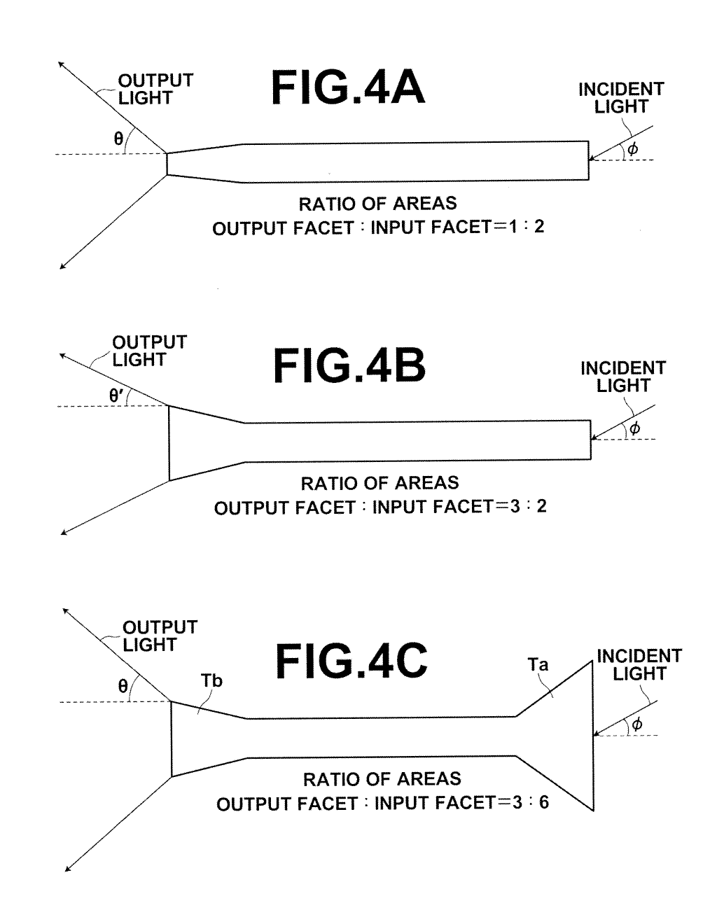

The reason for the above is as follows. FIG. 4A is a schematic diagram that illustrates an incident angle φ of illumination light with respect to an optical fiber and an angle of spread θ of output light in a conventional optical fiber having a tapered portion that becomes thinner toward the end. In the case that the core diameter at the output end is increased from the viewpoint of laser beam safety as illustrated in FIG. 4B, the light emitting area at the output end increases. However, the angle of spread decreases due to conservation of etendue. By increasing the core diameter at the light input end such that the ratio of the areas of the ends (light output end:light input end) is equivalent to the case illustrated in FIG. 4A, as illustrated in FIG. 4C, the angle of spread will theoretically be preserved because the ratio between areas is preserved.

The forceps outlet 27 is connected to the forceps opening 17 via a forceps channel 35. The forceps channel 35 is a cylindrical member formed by resin, for example. In the case that a diseased site is to be cut open while under observation with the endoscope, an electric scalpel 36 (high frequency scalpel), which is a treatment implement, is inserted into the forceps channel 35 through the forceps opening 17.

The processing apparatus 11 is equipped with a socket 40 that engages the communications connector 19a of the universal cord 15. The socket 40 is assembled into a housing 41 via an insulator (not shown) to electrically isolate the main body of the processing apparatus 11 and the connector 19. The housing 41 is grounded to earth. When the communications connector 19a is engaged with the socket 40, the CCD 30 is connected to the timing/driver circuit 42 and the DSP 43.

The timing/driver circuit 42 generates control signals (clock pulses) in response to commands from a CPU 44, and inputs the control signals to the CCD 30 via the signal cables 32. The control signals control the timings at which accumulated electric charges are read out from the CCD 30, the shutter speed of an electronic shutter of the CCD 30, etc. The DSP 43 administers color separation, color interpolation, gain correction, white balance adjustment, gamma correction, etc., on the image signals which are input via the signal cables 32, to generate image data. The image data are converted into analog signals by a digital/analog converter 45, and displayed on the monitor 20 as an endoscope image.

The light source apparatus 12 is equipped with: a light source 50; a light source driver 51; a diaphragm adjusting mechanism 52; an iris driver 53; and a CPU 54 that controls the other components. The light source 50 is turned ON and OFF according to control exerted by the light source driver 51, to irradiate illumination light toward a focusing lens 55 provided forward of the light source 50. Examples of the light source 50 include: a xenon lamp; a halogen lamp; an LED (Light Emitting Diode); a fluorescent light emitting element; and an LD (Laser Diode). The light source 50 is selected as appropriate according to the type of endoscope image (a visible image, a fluorescence image, etc.) is to be obtained, that is, the wavelength of light to be utilized.

The diaphragm adjusting mechanism 52 is provided between the light source 50 and the focusing lens 55, and adjusts the amount of illumination light such that the endoscope images obtained by the CCD 30 are of a substantially uniform brightness. The diaphragm adjusting mechanism 52 is equipped with: diaphragm wings that change the diameter (aperture diameter) of an aperture that the illumination light passes through; and a motor for driving the diaphragm wings. The iris driver 53 opens and closes the diaphragm wings of the diaphragm adjusting mechanism 52, to change the area that the illumination light passes through thereby adjusting the amount of illumination light that enters the light guide 34.

The flexible tube portion 13a of the probe 13 is constituted by: a flexible helical tube; a net that prevents stretching of the helical tube; and an outer layer which is a resin coating on the net. A plurality of the signal cables 32, the forceps channel 35, and a plurality of optical fibers F that constitute the light guide 34 are fed through the interior of the flexible tube portion 13a parallel to and in proximity to each other.

Next, the operation of the endoscope system 2 configured as described above will be described. When the electronic endoscope 10 is connected to the processing apparatus 11, the CCD 30 is connected to the timing/driver circuit 42 and the DSP 43. When the power source of the endoscope system 2 is switched ON, the processing apparatus 11 and the light source apparatus 12 initiate operations. The light source 50 of the light source apparatus 12 is turned ON, and illumination light is emitted toward the focusing lens 55. The illumination light is guided by the focusing lens 55 to the input end of the light guide 34, then guided to the tip portion 13c of the electronic endoscope 10.

After the probe portion 13 of the electronic endoscope 10 is inserted within a body cavity and the illumination light guided by the light guide 34 propagates within the light diffusing element 33, the illumination light is irradiated onto a portion to be observed. Then, an image of a portion to be observed, which is illuminated by the illumination light, is obtained by the CCD 30. The image signals output from the CCD 30 undergoes various processes in the analog processing circuit of the circuit board 31, then are input to the DSP 43 of the processing apparatus 11 via the signal cables 32. The DSP 43 administers various signal processes onto the input image signals, and generates image data. The generated image data are displayed as an endoscope image on the monitor 20 via the D/A converter 45.

In the case that treatment of a diseased site is necessary under endoscope observation, the electric scalpel 36 is inserted into the forceps channel 35 through the forceps opening 17. Then, the tip of the electric scalpel 36, to which high frequency electric current is applied, is caused to contact the diseased site to cut and cauterize the diseased site.

As described above, the light guide for an endoscope of the present invention and the endoscope equipped with the light guide are equipped with an optical fiber. The optical fiber has a light input side tapered portion and a light output side tapered portion. The light input side tapered portion is a predetermined portion of the optical fiber that includes an input end into which the illumination light enters the optical fiber, and the core of the optical fiber has a shape that becomes thicker toward the input end in the light input side tapered portion. The light output side tapered portion is a predetermined portion of the optical fiber that includes an output end through which the illumination light is output, and the core of the optical fiber has a shape that becomes thicker toward the output end in the light output side tapered portion. The optical fiber is characterized by the core diameter at the input end being greater than the core diameter at the output end. Thereby, laser beam intensity per visual angle can be reduced by increasing the light emitting area at the output end of the optical fiber. Meanwhile, the input end of the optical fiber is formed as a tapered shape that becomes thicker toward the input end, which has a greater core diameter than that of the output end. Therefore, the angle of spread at the output end can be increased, according to the conservation of etendue. As a result, an increase in angle of spread can be realized without tapering the optical fiber such that the optical fiber becomes thinner at the end thereof. At the same time, reduction of laser beam intensity per visual angle at an output end of the optical fiber can be realized by forming a light source having a large light emitting area.

Further, based on the effects described above, the light guide for an endoscope of the present invention and the endoscope equipped with the light guide exhibit the advantageous effects that an illuminated region is expanded and the safety standard class of laser light is reduced.

In addition, it is possible to produce the light guide for an endoscope and the endoscope equipped with the light guide that exhibit the aforementioned operational effects by the first and second methods for producing a light guide for an endoscope of the present invention.

### (Design Modifications to the Light Guide for an Endoscope)

A case in which the light guide for an endoscope of the present invention is applied to a flexible scope was described above. However, the present invention is not limited to this configuration, and the light guide for an endoscope of the present invention may be applied to a rigid scope.

### Embodiments

Embodiments of the light guide for an endoscope of the present invention will be described below.

### Embodiment 1

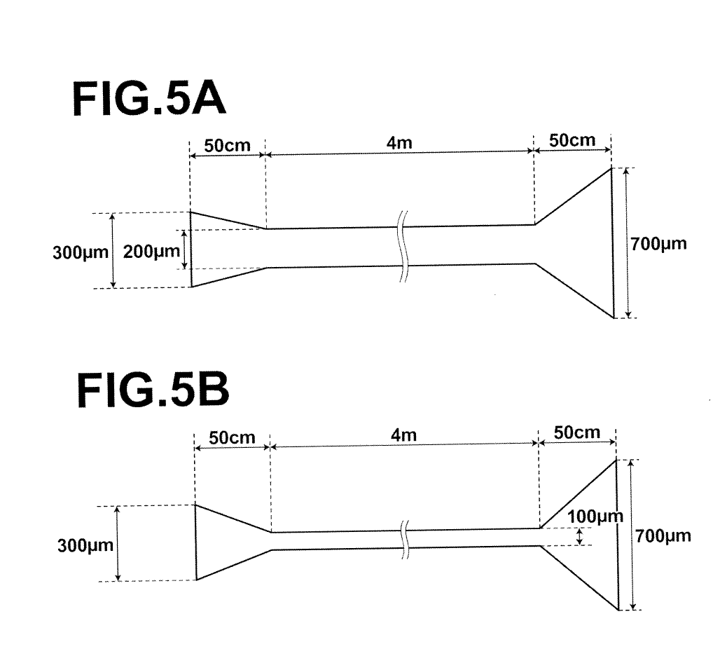

Two quartz multimode optical fibers having a numerical aperture NA of 0.22 at the linear portions thereof, core diameters of 200 μm, cladding diameters of 240 μm, and total lengths of 2.5 m, onto which tapering processes were administered such that first ends thereof were tapered shapes that become thicker toward the end, were prepared. The tapering process was administered to one of the two optical fibers such that tapering began at a position 50 μm from the first end and the core diameter at the first end was 700 μm. The tapering process was administered to the other of the two optical fibers such that tapering began at a position 50 cm from the first end and the core diameter at the first end was 300 μm. Thereafter, the thin diameter ends (the ends on which the tapering processes were not administered) of the first optical fiber and the second optical fiber were fused. Thereby, an optical fiber strand having a light input side tapered portion that becomes thicker toward the end with a core diameter of 700 μm at the end and a light output side tapered portion that becomes thicker toward the end with a core diameter of 300 μm as illustrated in FIG. 5A was produced. Then, the optical fiber strand was utilized to produce a light guide for an endoscope. Note that in FIGS. 5A and 5B, only the core portions of optical fibers are illustrated for the sake of convenience.

At this time, the visual angle α of the light output end of the optical fiber as viewed from a position 100 mm away was 3 mrad. Accordingly, the AEL (Accessible Emission Limit) of this light guide for an endoscope can be approximately twice that calculated for a case in which a point light source having a visual angle of 1.5 mrad is employed. This means that approximately twice the luminance is allowable in the case that the laser class is 1, for example. The NA at the light input side is 0.22·200 μm/700 μm=0.06, and the NA at the light output side is 0.22·200 μm/300 μm=0.15, based on conservation of etendue. Therefore, it can be understood that the spread angle of the beam at the light output end is greater than the incident angle at the light input end.

### Embodiment 2

Two quartz multimode optical fibers having a numerical aperture NA of 0.5 at the linear portions thereof, core diameters of 100 μm, cladding diameters of 115 μm, and total lengths of 2.5 m, onto which tapering processes were administered such that first ends thereof were tapered shapes that become thicker toward the end, were prepared. The tapering process was administered to one of the two optical fibers such that tapering began at a position 50 cm from the first end and the core diameter at the first end was 700 μm. The tapering process was administered to the other of the two optical fibers such that tapering began at a position 50 cm from the first end and the core diameter at the first end was 300 μm. Thereafter, the thin diameter ends (the ends on which the tapering processes were not administered) of the first optical fiber and the second optical fiber were fused. Thereby, an optical fiber strand having a light input side tapered portion that becomes thicker toward the end with a core diameter of 700 μm at the end and a light output side tapered portion that becomes thicker toward the end with a core diameter of 300 μm as illustrated in FIG. 5B was produced. Then, the optical fiber strand was utilized to produce a light guide for an endoscope.

At this time, the visual angle α of the light output end of the optical fiber as viewed from a position 100 mm away was 3 mrad. Accordingly, the AEL (Accessible Emission Limit) of this light guide for an endoscope can be approximately twice that calculated for a case in which a point light source having a visual angle of 1.5l mrad is employed. In addition, a laser beam having a total intensity of two times or greater may be utilized. The NA at the light input side is 0.5·100 μm/700 μm=0.07, and the NA at the light output side is 0.5·100 μm/300 μm=0.15, based on conservation of etendue. Therefore, it can be understood that the spread angle of the beam at the light output end is greater than the incident angle at the light input end.

What is claimed is:

1. A light guide for an endoscope equipped with an optical fiber for guiding illumination light to a portion to be observed, the optical fiber comprising:
a light input side tapered portion; and
a light output side tapered portion;
the light input side tapered portion being a predetermined portion of the optical fiber that includes an input end into which the illumination light enters the optical fiber, and the core of the optical fiber having a shape that becomes thicker toward the input end in the light input side tapered portion;
the light output side tapered portion being a predetermined portion of the optical fiber that includes an output end through which the illumination light is output, and the core of the optical fiber having a shape that becomes thicker toward the output end in the light output side tapered portion; and
the core diameter at the input end being greater than the core diameter at the output end.

2. A light guide for an endoscope as defined in claim 1, wherein:
the ratio of the core diameter at the input end with respect to the core diameter at the output end is within a range from 2 to 3.

3. A light guide for an endoscope as defined in claim 1, wherein:
the core diameter at the input end is within a range from 300 μm to 6000 μm, and the core diameter at the output end is within a range from 150 μm to 4000 μm.

4. A light guide for an endoscope as defined in claim 1, wherein:
the optical fiber is a multimode optical fiber.

5. An endoscope, comprising:
a light guide for an endoscope as defined in claim 1;
a light source for generating the illumination light, connected to an input side of the light guide for an endoscope; and
an imaging section, for receiving light generated at the portion to be observed due to irradiation of the illumination light guided by the light guide for an endoscope and for imaging the portion to be observed.

6. A method for producing a light guide for an endoscope that guides illumination light to a portion to be observed, comprising:
preparing a first optical fiber having a first tapered portion at a first end thereof and a second optical fiber having a second tapered portion at a first end thereof;
fusing a second end of the first optical fiber and a second end of the second optical fiber to form a third optical fiber having the first tapered portion and the second tapered portion; and
placing the third optical fiber into a probe portion of an endoscope such that the second tapered portion is an input side of the illumination light and the first tapered portion is an output side of the illumination light;
the first tapered portion being a predetermined portion of the first optical fiber that includes the first end thereof, and the core of the optical fiber having a shape that becomes thicker toward the first end of the first optical fiber in the predetermined portion;
the second tapered portion being a predetermined portion of the second optical fiber that includes the first end thereof, and the core of the optical fiber having a shape that becomes thicker toward the first end of the second optical fiber in the predetermined portion; and
the core diameter at the first end of the second optical fiber being greater than the core diameter of the first end of the first optical, fiber.

7. A method for producing a light guide for an endoscope as defined in claim 6, wherein:
the ratio of the core diameter at the first end of the first optical fiber with respect to the core diameter at the first end of the second optical fiber is within a range from 2 to 3.

8. A method for producing a light guide for an endoscope as defined in claim 6, wherein:
the core diameter at the first end of the second optical fiber is within a range from 300 μm to 6000 μm, and the core diameter at the first end of the first optical fiber is within a range from 150 μm to 4000 μm.

9. A method for producing a light guide for an endoscope that guides illumination light to a portion to be observed, comprising:
processing a predetermined portion of an optical fiber that includes a first end of the optical fiber to be tapered such that the core of the optical fiber in the predetermined portion becomes thicker toward the first end;
processing a predetermined portion of the optical fiber that includes a second end of the optical fiber to be tapered such that the core of the optical fiber in the predetermined portion becomes thicker toward the second end and such that the core diameter at the second end is greater than the core diameter at the first end; and
placing the optical fiber within a probe portion of an endoscope such that the end having the greater core diameter is a light input side of the illumination light and the end having the smaller core diameter is an output side of the illumination light.

10. A method for producing a light guide for an endoscope as defined in claim 9, wherein:
the ratio of the core diameter at the second end of the optical fiber with respect to the core diameter at the first end of the optical fiber is within a range from 2 to 3.

11. A method for producing a light guide for an endoscope as defined in one of claim 9, wherein:
the core diameter at the second end of the optical fiber is within a range from 300 μm to 6000 μm, and the core diameter at the first end of the optical fiber is within a range from 150 μm to 4000 μm.
